(19) United States
(12) Patent Application Publication
(10) Pub. No.: US 2014/0058277 A1
(43) Pub. Date: Feb. 27, 2014

(54) BLOOD FLOW RATE MEASUREMENT SYSTEM

(71) Applicants: Seng Sing Tan, Singapore (SG); Cheong Ming Lam, Singapore (SG)

(72) Inventors: Seng Sing Tan, Singapore (SG); Cheong Ming Lam, Singapore (SG)

(73) Assignee: Nanyang Polytechnic, Singapore (SG)

(21) Appl. No.: 14/003,748

(22) PCT Filed: Mar. 9, 2011
(86) PCT No.: PCT/SG2011/000093

§ 371 Date: Nov. 8, 2013

(51) Int. Cl. A61F 7/12; A61B 5/0215; A61B 5/00; A61B 5/026

(52) U.S. Cl. CPC A61F 7/12; A61B 5/026; A61B 5/0215; A61B 5/6853
USPC 600/487; 600/505; 607/106

(57) ABSTRACT

The present invention discloses a heat exchanging assembly with a body adaptable to a catheter for measuring blood flow rate with a supply system for providing the heat exchanging medium to the heat exchanging assembly at a constant temperature and constant flow rate. The heat exchanging assembly comprises a heat exchanging element, a plurality of temperature sensors, a sensor lumen and an inflation lumen.

### FIELD OF THE INVENTION

This invention relates to a system for measuring blood flow rate. More particularly, this invention relates to a heat exchanging assembly in the system that is adaptable to a catheter for measuring blood flow rate. Still more particularly, this invention relates to a system for providing a heat exchanging medium to the heat exchanging assembly that ensures that the heat exchanging medium provided to the exchanging assembly is able to be supplied consistently at a constant temperature.

### PRIOR ART

Coronary artery heart disease is a condition whereby blood flowing in the coronary arteries that supply the heart with oxygenated blood is obstructed. An example of such a disease is atherosclerosis, which causes the narrowing, and hardening of the arteries. Due to this blockage in the arteries, the rate of flow of blood in the blood vessel will be greatly decreased.

Angioplasty, a mechanical technique used to enlarge narrowed or obstructed blood vessels, is commonly used to treat heart diseases such as atherosclerosis. In an angioplasty procedure, a balloon catheter is guided to locations in the blood vessel which are obstructed or narrowed using a guide wire. The balloon at the front of the catheter is then inflated to crush the fatty deposits thus opening up the blood vessels to increase the blood flow rate. The balloon is then deflated and the catheter is withdrawn. The blood flow rate of the patient before and after the angioplasty procedure has to be measured in order to judge whether the angioplasty procedure has been successful. Therefore, the procedure requires a method or system that accurately measures the blood flow rate of a patient without introducing any adverse side effects that may further deteriorate a patient's condition.

Commercially available blood flow rate measurement devices incorporate a heating element in the catheter. These devices indirectly heat a segment of the blood with the heating element that is typically an electric resistance heater. The temperature deviation of the blood is then monitored as a function of time at a location downstream from the location at which the heating element is placed. The measured thermo-dilution curve is then used to determine the blood flow rate of the patient. Publications which describe such devices include U.S. Pat. No. 4,785,823 and U.S. Pat. No. 5,509,424. These devices are disadvantageous to the patient as the heating of the blood may provoke a fever-like response from the patient and are not appropriate for use on patients having fever.

As the heating of segments of blood is disadvantageous, other proposed methods have been used and remain popular. U.S. Pat. No. 4,841,981, U.S. Pat. No. 4,901,734 and U.S. Pat. No. 6,986,744 propose blood flow rate measurement methods that involve cooling segments of blood. The described systems propose a method in which an indicator liquid is injected into the blood stream of the patient. The indicator liquid that is injected is at a temperature that is lower than the blood temperature of the patient. The cardiac output of the patient is then measured using a thermo-dilution method. A problem with these methods is that these methods involve introducing foreign objects, e.g. the indicator liquid, into the patient's system. The injection of the foreign objects may result in the patient having an adverse reaction such as an allergic reaction. Thus, these proposed methods are not ideal methods for determining the blood flow of a patient.

An apparatus for measuring blood flow rate that does not involve heating segments of blood and does not involve injecting foreign objects into a patient's blood stream is disclosed in PCT Application No. PCT/SG2008/000234 titled “Process and Apparatus for Determining Blood Flow Rate or Cardiac Output”. This publication discloses an apparatus comprising a flexible closed-loop heat transfer system with temperature sensors for monitoring the temperature of a heat exchange medium in the closed loop system. In the described system, the temperature of the heat exchanging medium is measured before the medium enters a heat transfer element as the temperature of the heat exchanging medium supplied to the heat transfer element is uncontrollable. Additionally, a guide wire catheter is needed to position the heat transfer element in the blood vessel of the patient. It is a problem that this disclosure does not teach guiding or sensing means to position the catheter at the desired location in the pulmonary artery of the patient's heart.

Therefore, there is a need in the art for an apparatus or method for determining the blood flow rate of a patient whereby only the temperature of the heat exchanging medium flowing out from the heat transfer element is the essential parameter to be measured and whereby the temperature of the heat exchanging medium introduced into the heat transfer element is maintained at a constant level, with minimal temperature fluctuations to ensure repeatable and accurate measurements. There is also a need in the art for a heat exchanging assembly adaptable to catheters to provide guidance for the catheter during placement in the blood vessel.

### SUMMARY OF THE INVENTION

The above and other problems are solved and an advance in the art is made by methods and systems in accordance with this invention. A first advantage of systems and methods in accordance with this invention is that the temperature of the heat exchanging medium does not need to be measured before the heat exchanging medium is introduced into the heat exchanging element. A second advantage of systems and methods in accordance with this invention is that means for guiding the catheter and means for sensing the position of the catheter in the blood stream of a patient are provided. A third advantage of systems and methods in accordance with this invention is that the temperature of the heat exchanging medium is maintained at a constant temperature with minimal temperature fluctuations prior to being introduced in a heat exchanging element. Thus, the blood flow rate measurements are ensured to be repeatable.

In accordance with some embodiments of the invention, a system for maintaining a heat exchanging medium at a constant temperature and at a constant flow rate is configured in the following manner. A reservoir connects to a pump via a first path. The pump pumps a heat exchanging medium out of the reservoir to a cooling apparatus connected to the pump by a second path. The cooling apparatus is also connected to the reservoir by a third path. The heat exchanging medium pumped out of the reservoir is cooled by the cooling apparatus and flows out of the apparatus via the third path. A valve located in the third path selectively connects the cooling apparatus to the reservoir and heat transfer mechanism. When the valve is in a first configuration, the cooled heat exchanging medium flows along the third path to the reservoir. When the valve is in a second configuration, the flow of the cooled heat exchanging medium is directed to a fourth path which is connected to a catheter. A fifth path then connects an outlet of the catheter to the reservoir and the cooled heat exchanging medium flows from the catheter to the reservoir along the fifth path.

In accordance with some embodiments of the invention, the valve is located proximate to the cooling apparatus to ensure that the cooled heat exchanging medium flowing to the catheter along the fourth path is at a stabilised cooled temperature with minimal temperature fluctuations.

In accordance with some embodiments of the invention, the cooling apparatus is an active cooler such as a fan or a peltier cooler; or a passive cooler such as a thermal heat sink. In accordance with some embodiments of the invention, the pump is an electric pump connected to an alternating current or direct current power supply to circulate the heat exchanging medium in the system.

In accordance with some embodiments of the invention, the first, second, third, fourth and fifth paths are formed of thermo-insulated tubing that are flexible and the heat exchanging medium may be some liquid such as water, saline or some water-saline mixture.

In accordance with some embodiments of the invention, a heat exchanging assembly with a body adaptable to a catheter for measuring blood flow rate is configured in the following manner. A heat exchanging element which is here on defined as a heat exchanging coil has a first end and a second end. The first end of the heat exchanging coil is an inlet for a heat exchanging medium and the second end of the heat exchanging coil is an outlet for the heat exchanging medium. A first temperature sensor in the heat exchanging assembly monitors the temperature of the heat exchanging medium in the heat exchanging coil. A second temperature sensor in the heat exchanging assembly monitors the temperature of a liquid, such as blood, flowing over the heat exchanging coil. An inflation lumen has a first end and second end. The first end is connected to an inflatable device located on a head-end of the catheter and the second end is connected to an inflation apparatus. A sensor lumen in the assembly has a first end and a second end. The first end of the sensor lumen extends to an opening at the distal end of the catheter and the second end is connected to an external monitoring apparatus. The sensor lumen provides a pressure communication channel. Hence, the treating physician can closely monitor the pressure in the blood stream during the insertion process.

In accordance with some embodiments of the invention, the external monitoring apparatus is used to measure the variation in blood pressure at the opening at the distal end of the catheter. In further embodiments of the invention, the heat exchanging assembly may include a supply system for providing the heat exchanging medium to the first end of the heat exchanging coil at a constant temperature and flow rate.

In accordance with some embodiments of the invention, the heat exchanging assembly comprises a third temperature sensor to monitor the temperature of the heat exchanging medium in the assembly.

### BRIEF DESCRIPTION OF THE DRAWINGS

The above advantages and features of a method and system in accordance with this invention are described in the following detailed description and are shown in the drawings:

FIG. 1 illustrating a schematic view of a heat exchanging assembly adaptable to a catheter for measuring blood flow rate in accordance with an embodiment of this invention;

FIG. 2 illustrating an enlarged view of a distal end of the catheter in accordance with an embodiment of this invention;

FIG. 3 illustrating an enlarged view of a heat exchanging coil in accordance with an embodiment of this invention;

FIG. 4 illustrating a longitudinal cross-sectional view of a catheter tube and a heat exchanging coil in accordance with an embodiment of this invention;

FIG. 5 illustrating a cross sectional view along line C-C′ as shown in FIG. 4;

FIG. 6 illustrating an enlarged longitudinal cross sectional view of area B′ as shown in FIG. 4;

FIG. 7 illustrating an enlarged longitudinal cross sectional view of area A′ as shown in FIG. 4;

FIG. 8 illustrating the temperature difference versus the blood flow rate measurements obtained from an embodiment of this invention;

FIG. 9 illustrating the blood flow rate versus time measurements obtained from an embodiment of this invention; and

FIG. 10 illustrating a heat exchanging medium supply system in accordance with an embodiment of this invention.

### DETAILED DESCRIPTION

This invention relates to a system for measuring blood flow rate. More particularly, this invention relates to a heat exchanging assembly in the system that is adaptable to a catheter for measuring blood flow rate. Still more particularly, this invention relates to a system for providing a heat exchanging medium to the heat exchanging assembly that ensures that the heat exchanging medium provided to the exchanging assembly is at a constant temperature.

The present invention relates to a system for measuring blood flow rate. In the system, the heat exchanging medium flowing in the system never mixes or combines with the patient's blood. For purposes of this discussion, the heat exchanging medium can be any type of liquid which can absorb or dissipate heat. Examples of heat exchanging medium include, but are not limited to, water and a water/saline solution.

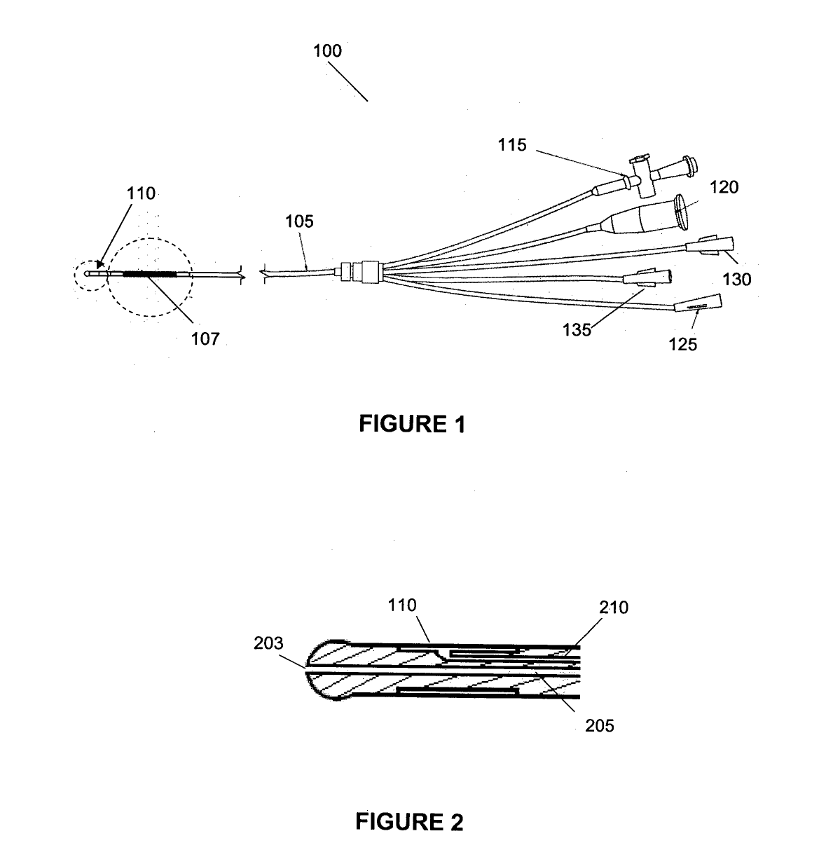

FIG. 1 illustrates a schematic view of catheter 100 that incorporates an embodiment of this invention. Catheter 100 includes catheter tube 105 having a proximal end and a distal end, heat exchanging coil 107, inflatable device 110 located at the distal end of catheter tube 105, inflation port 115, temperature port 120, sensor port 125, inlet port 130 and outlet port 135. Inflation port 115 is connected to an inflation apparatus (not shown). The inflation apparatus inflates and/or deflates inflatable device 110. In accordance with the shown embodiment, inflatable device 110 is an inflatable rubber balloon or a similar inflatable device made from bio-compatible material and the inflation apparatus is a syringe or a similar device. Temperature port 120 is connected to a temperature measurement apparatus (not shown). The temperature measurement apparatus monitors temperature sensors and displays the measured temperature. Inlet port 130 connects to a heat exchanging supply system and introduces a heat exchanging medium into heat exchanging coil 107. Outlet port 135 discharges the heat exchanging medium from heat exchanging coil 107 to an outer source. Sensor port 125 connects opening 203 (shown in FIG. 2) which is located at a distal end of catheter tube 105 via sensor lumen 205 (shown in FIG. 2) to external monitoring equipment (not shown). The external monitoring equipment is used to detect parametric changes at the distal tip of catheter 105.

When in use, catheter 100 is inserted into a blood vessel and heat exchanging coil 107 is positioned at a desired location in the circulatory system. Inflatable device 110, which may be inflated using a gaseous medium, assists in guiding the distal end of catheter tube 105 to the desired location. One advantage of using inflatable device 110 to guide catheter tube 105 is the pliable nature of inflatable device 110. Thus, inflatable device 110 assists in reducing trauma and damage to the blood vessels as catheter tube 105 is being guided to the desired location. Once catheter tube 105 is positioned at the desired location, inflatable device 110 may then be deflated using the inflation apparatus. The external monitoring equipment connected to sensor port 125 assists in guiding catheter tube 105 to the desired location by detecting parametric changes. In the shown embodiment, the external monitoring equipment is a pressure monitor that detects changes in blood pressure, at opening 203, as catheter tube 105 moves through different parts in the circulatory system.

FIG. 2 illustrates an enlarged view of the distal end of catheter tube 105 and shows inflatable device 110 at the distal end of catheter tube 105. In accordance with the shown embodiment, inflatable device 110 is an inflatable rubber balloon around the circumference of catheter tube 105 proximate the distal end. One skilled in the art will appreciate that inflatable device 110 may be a balloon, an annular ring, inflatable bladders or other similar inflatable device made from biocompatible material that may be attached to any portion of catheter tube 105 without departing from this invention. Furthermore, one skilled in the art will recognizer that more than one, inflatable device may be incorporated into catheter 105 without departing from this invention. Examples of biocompatible materials that may be used in inflatable device 110 include, but are not limited to, silicon and polyester. As shown in FIG. 2, inflatable device 110 is connected to inflation port 115 through inflation lumen 210. As shown in FIG. 11, inflatable device 110 expands when a gaseous substance is pumped in through inflation port 115. Sensor lumen 205 has one end connected to sensor port 125 and another end extending to opening 203, located at the distal end of catheter tube 105. Sensor lumen 205 fills with blood or other bodily fluid through opening 203 when in use. External monitoring equipment (not shown) connected to sensor port 125 detects parametric changes at opening 203 by performing measurements on the liquid in sensor lumen 205. In one embodiment, the liquid that fills sensor lumen 205 is the patient's blood. In this embodiment, the external monitoring equipment connected to sensor port 125 measures the blood pressure at opening 203.

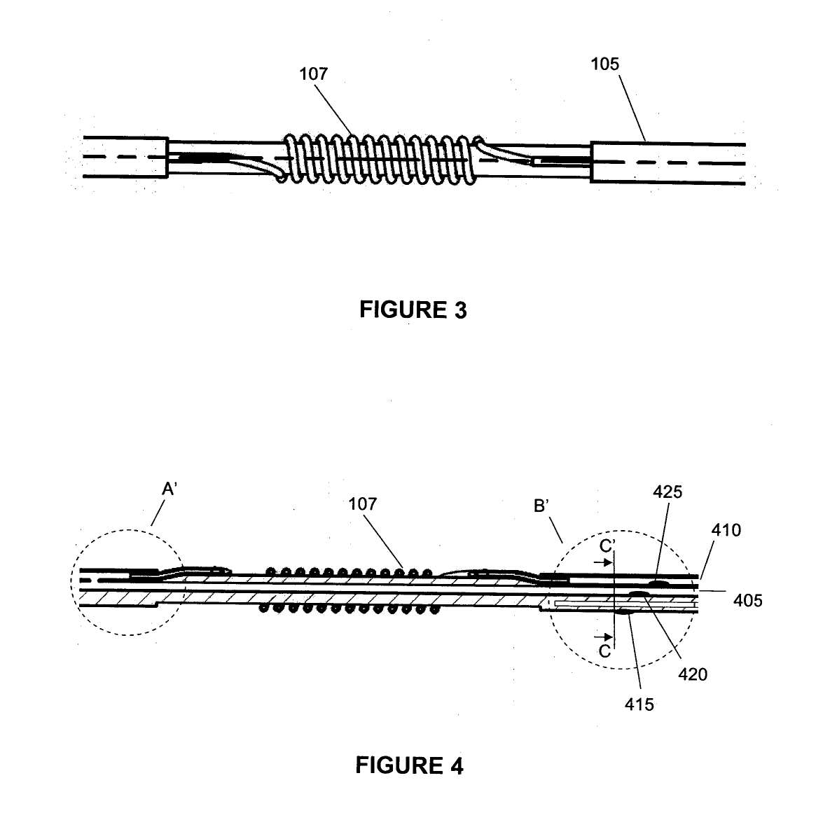

FIG. 3 illustrates an enlarged view of heat exchanging coil 107 in accordance with an embodiment of the present invention. Heat exchanging coil 107 is wrapped around catheter tube 105 in a helical configuration. The helical configuration maximises the exposed surface area of heat exchanging coil 107 inside the blood vessels when the invention is in use. The number of turns in the helical configuration of heat exchanging coil 107 is left as a design choice. Generally, the number of turns is proportional to a desired exposed surface area for facilitating the heat exchange. Heat exchanging coil 107 may be made from biocompatible and/or thermo conductive materials. Examples of thermo conductive materials, include but are not limited to, stainless steel and titanium. However, one skilled in the art will recognize that other types of material may be used without departing from this invention.

FIG. 4 illustrates a longitudinal cross-sectional view of catheter tube 105 and heat exchanging coil 107 in accordance with one embodiment of the present invention. The heat exchanging medium flows into heat exchanging coil 107 at inlet end 405. After the heat exchanging medium passes through the helical configuration of heat exchanging coil 107, the heat exchanging medium flows out of heat exchanging coil 107 through outlet end 410. The exact heat exchanging medium used is left as a design choice. However, for health reasons, heat exchanging medium is preferably chosen to be a non-poisonous liquid in case leakages occur.

External temperature sensor 415 is located proximate to the surface of catheter tube 105, inlet temperature sensor 420 is located proximate inlet end 405 and outlet temperature sensor 425 is located proximate outlet end 410. External temperature sensor 415 measures the temperature of the external elements in the blood vessels such as the temperature of the blood flowing over catheter tube 105. Inlet temperature sensor 420 measures the temperature of the heat exchanging medium as the medium flows into heat exchanging coil 107. Outlet temperature sensor 425 measures the temperature of the heat exchanging medium as the medium flows out from heat exchanging coil 107. In certain embodiments of this invention, inlet temperature sensor 420 may be omitted if the temperature of the heat exchanging medium before the medium enters heat exchanging coil 107 is manageable. Preferably, temperature sensors 415-425 are contact temperature sensors such as thermistors. However, one skilled in the art will recognize that other types of temperature sensors can be used without departing from this invention.

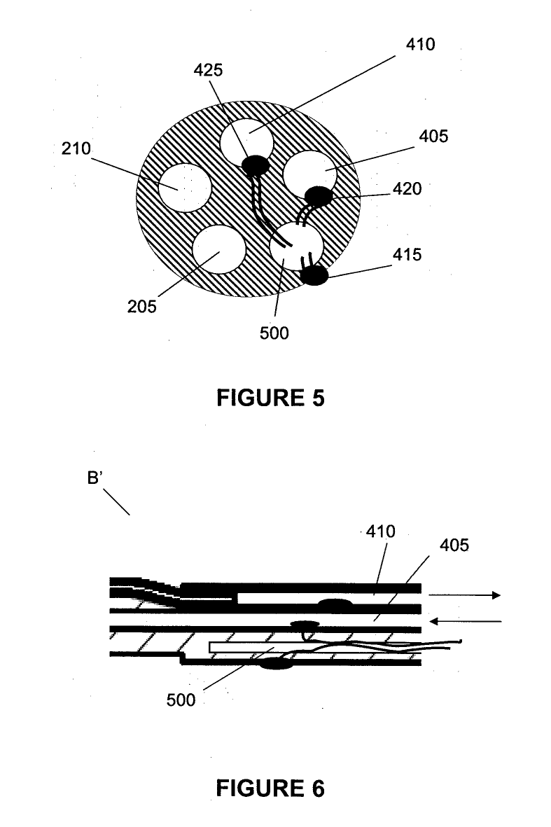

FIG. 5 illustrates a cross sectional view of line C-C′ as shown in FIG. 4. The cross sectional view illustrates external temperature sensor 415 located proximate temperature lumen 500. Temperature lumen 500 has one end connected to temperature port 120. Sensor lumen 205 has one end connected to sensor port 125. Inflation lumen 210 has one end connected to inflation port 115. Temperature lumen 500 provides means for temperature sensors 415-425 to be electrically connected to the temperature measurement apparatus. FIGS. 4 and 5 illustrate that under normal use, the heat exchanging medium is contained within the heat exchanging assembly and does not mix or flow into the blood vessel at any time.

FIG. 6 illustrates an enlarged longitudinal cross sectional view of B′ as shown in FIG. 4. The direction of the flow of the heat exchanging medium is shown. The cooled heat exchanging medium flows into heat exchanging coil 107 at inlet end 405 and flows out from heat exchanging coil 107 through outlet end 410.

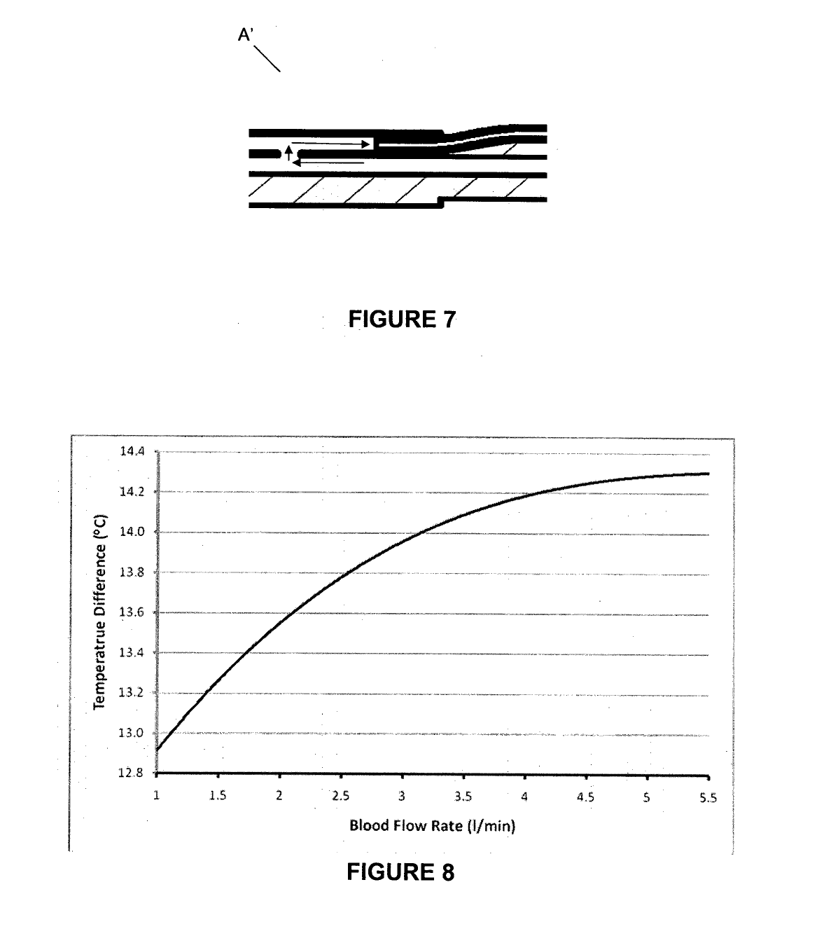

FIG. 7 illustrates an enlarged longitudinal cross sectional view of A′ as shown in FIG. 4. The direction of the flow of the heat exchanging medium at the distal end of catheter tube 105 is shown in FIG. 7.

A preliminary experiment has been conducted using a heat-transfer assembly as shown in FIGS. 1-7. The preliminary experiment confirmed the application of the relationship derived through theoretical analysis based on the following equations (1)-(3).

Q_{f}=m_{f}C_{f} (T_{fi}−T_{fo}) (1)

Q_{b}=m_{b}C_{b}(T_{bi}−T_{bo}) (2)

Q=UA(LMTD) (3)

where,
Q_{f}=heat transfer rate to the heat exchanging medium in the heat exchanging coil;
Q_{b}=heat transfer rate from the blood flowing over the heat exchanging coil;
Q=heat transfer rate through the heat exchanging coil;
m_{f}=mass flow rate of the heat exchanging medium in the heat exchanging coil;
m_{b}=mass flow rate of the blood flowing over the heat exchanging coil;
C_{f}=specific heat capacity of the heat exchanging medium in the heat exchanging coil;
C_{b}=specific heat capacity of the blood flowing over the heat exchanging coil;
T_{fi}=inlet temperature of the heat exchanging medium in the heat exchanging coil;
T_{fo}=outlet temperature of the heat exchanging medium in the heat exchanging coil;
T_{bi}=inlet temperature of the blood over the heat exchanging coil;
T_{bo}=outlet temperature of the blood over the heat exchanging coil;
UA=(heat transfer coefficient) x (surface area of the heat exchanging coil);
LMTD=Log Mean Temperature Difference
   LMTD is a function of T_{fi}, T_{fo}, T_{bi}, and T_{bo} while UA could be assumed to be constant for a specific heat transfer system. Q could be equated to Q_{f} and Q_{b} and the mass flow rate of blood, m_{b}, could then be related to the outlet temperature of the heat exchange medium, T_{fo}, leaving the heat exchanging coil, by eliminating, T_{bo}, leaving the rest of the parameters, T_{fi}, T_{bi}, and UA which are predetermined in the expression. The results have demonstrated the relationship shown in FIG. 8. Thus, the cardiac output and the substantial change of the cardiac output could be determined by measuring the change in temperature of the heat exchanging medium leaving the heat exchanging coil using this relationship.

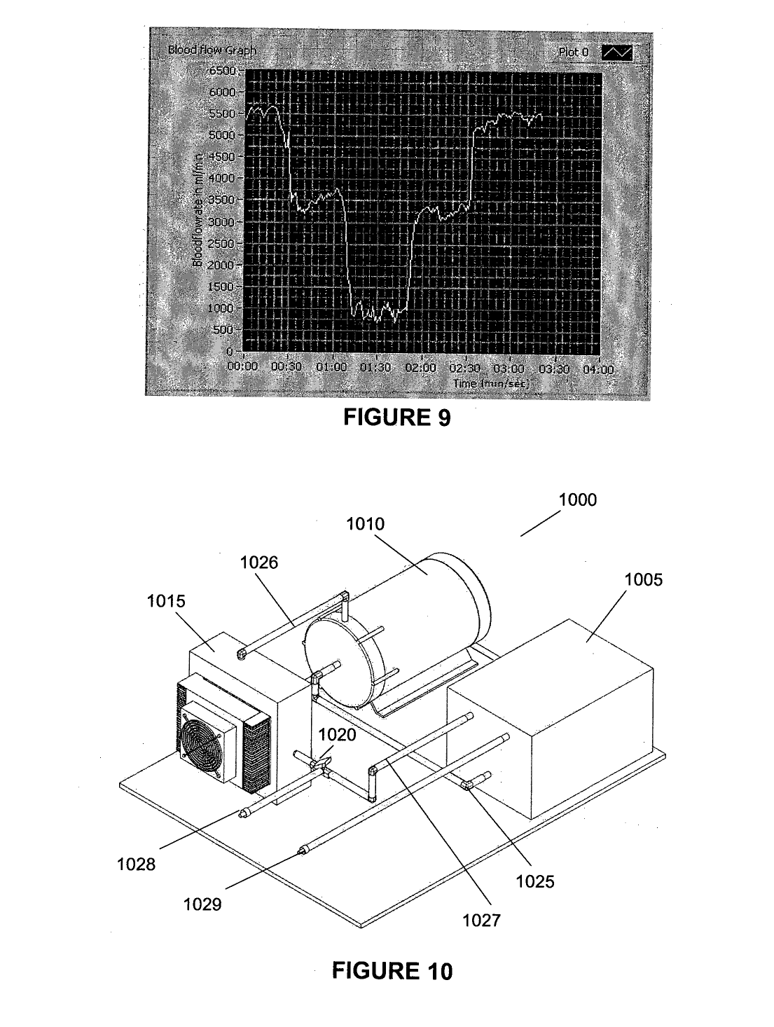

FIG. 9 shows the measured experimental results. The results show a range of blood flow rate between 5.5 to 1.0 litres per minute. FIG. 9 also shows that the variation in blood flow rate can be efficiently measured.

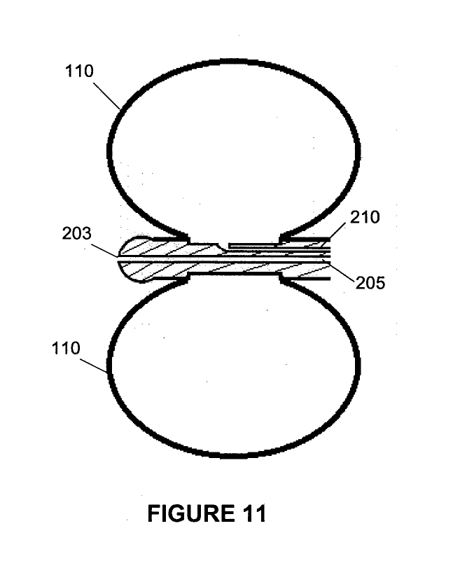

FIG. 10 illustrates supply system 1000 in accordance with an embodiment of this invention. Supply system 1000 provides heat exchanging medium at a constant temperature to an embodiment of the invention as shown in FIG. 1. Supply system 1000 includes reservoir 1005, pump 1010, cooling apparatus 1015, valve 1020, and paths 1025-1029. Path 1025 fluidly connects reservoir 1005 to pump 1010. Path 1026 fluidly connects pump 1010 to cooling apparatus 1015. Path 1027 fluidly connects cooling apparatus 1015 to reservoir 1005. Paths 1028 and 1029 are connected to inlet port 130 and outlet port 135 of catheter 100 respectively.

Valve 1020, fluidly connects path 1027 to path 1028 and has two configurations. In a first configuration, valve 1020 allows the heat exchanging medium to flow from cooling apparatus 1015 to reservoir 1005 along path 1027. Thus, a feedback loop to reservoir 1005 is formed to maintain the heat exchanging medium at the desired temperature when the medium is not being applied to the catheter. In a second configuration, the flow of the heat exchanging medium from cooling apparatus 1015 is redirected to path 1028 and into inlet port 130 of catheter 100. The heat exchanging medium then returns to reservoir 1005 through path 1029 which is connected to outlet port 135.

Supply system 1000 functions in the following manner. Reservoir 1005 is used to store a predetermined amount of heat exchanging medium for use with catheter 100. The capacity, size and material of reservoir 1005 are left as design choices to the user of an embodiment of this invention. When pump 1010 is operating, pump 1010 pumps the heat exchanging medium out from reservoir 1005 along path 1025. Pump 1010 then pumps the heat exchanging medium to cooling apparatus 1015 along path 1026.

Pump 1010 is preferably an electric pump connected to an alternating/ direct current power supply to circulate the heat exchanging medium in supply system 1000. However, one skilled in the art will recognize that other types of pumps may be used without departing from this invention. The flow rate of heat exchanging medium in supply system 1000 is determined by the pumping rate of pump 1010 and the pump rate is left as a design choice to a user of an embodiment of this invention. Preferably, pump 1010 maintains the flow rate between 10-15 ml/min.

Cooling apparatus 1015 is used to cool the heat exchanging medium passing through cooling apparatus 1015 to a predetermined temperature. Cooling apparatus 915 may be an active or a passive cooler, such as a cooler fan, a container filled with melting ice, a peltier cooler; or a thermal heat dispersing apparatus. Cooling apparatus 1015 may include a built in temperature sensor (not shown) to measure and display the temperature of the heat exchanging medium circulating in supply system 1000.

After passing through cooling apparatus 1015, the heat exchanging medium flows to reservoir 1005 along path 1027 in response to valve 1020 being in a first configuration. The heat exchanging medium is then pumped out from reservoir 1005 into pump 1010 and the heat exchanging medium repeats the entire cooling cycle as described above. The heat exchanging medium in supply system 1000 is maintained at a constant temperature and has a constant, stable flow rate while circulating through supply system 1000. The temperature of the heat exchanging medium is determined by cooling apparatus 1015 and the desired temperature is left is a design choice to a user of an embodiment of this invention.

In response to valve 1020 being in a second configuration, the heat exchanging medium flowing out from cooling apparatus 1015 is directed to path 1028 which is connected to inlet port 130. After the heat exchanging medium has passed through the entire length of heat exchanging coil 107, the heat exchanging medium flows into reservoir 1005 along path 1029 which is connected to outlet port 135.

Valve 1020 may comprise a stop valve or a check valve. Preferably, valve 1020 is a type of global valve having a cross flow configuration. Valve 1020 is not limited to stop valves or check valves; and it is envisioned that other types of valves may be used without departing from this invention.

Preferably, the temperature of the heat exchanging medium circulating in supply system 1000 should be between 0-5° C. Valve 1020 is located proximate cooling apparatus 1015 thus ensuring that when valve 1020 is in a second configuration, the temperature of the heat exchanging medium flowing to catheter 100 along path 1028 is approximately the temperature of the heat exchanging medium in supply system 1000. If valve 1020 is located at a position further away from cooling apparatus 1015, the temperature of the heat exchanging medium flowing into catheter 100 is not at the optimum temperature as the heat exchanging medium would have absorbed heat from external sources.

Paths 1025-1029 may comprise material such as thermo insulated flexible tubing. Thermo insulated tubing is preferable to ensure that the heat exchanging medium in supply system 1000 does not absorb heat from external sources while circulating prior to being redirected via valve 1020 to catheter 100. The overall size of supply system 1000 may be reduced when flexible tubing is used’, thus allowing reservoir 1005, pump 1010 and cooling apparatus 1015 to be placed in close proximity to one another.

When supply system 1000 is used to provide a heat exchanging medium to an embodiment of the invention as shown in FIGS. 1-7, a temperature sensor at the inlet port may be omitted as the temperature of the heat exchanging medium circulating in supply system 1000 is known. Therefore, when the heat exchanging medium is provided to catheter 100, there is no need for a temperature sensor at the inlet port. This results in a more efficient, repeatable, stable, and accurate blood flow rate measurement system.

1. A system for maintaining a heat-exchanging medium at a constant temperatur and at a constant flow rate comprising:
a reservoir;
a pump connected to said reservoir by a first path wherein said pump pumps heat-exchanging medium out of said reservoir;
a cooling apparatus connected to said pump by a second path and connected to said reservoir by a third path wherein said cooling apparatus cools said heat-exchanging medium pumped out of said reservoir;
a valve located in said third path wherein a first configuration of said valve allows said cooled heat-exchanging medium to flow along said third path to said reservoir and a second configuration of said valve redirects said cooled heat-exchanging medium from said third path to a fourth path;
a catheter, connected to said valve by said fourth path; and
a fifth path with a first end connected to said reservoir and a second end connected to said catheter wherein said cooled heat-exchanging medium flows from said catheter to said reservoir along said fifth path.

2. The system of claim 1 wherein said valve is located proximate said cooling apparatus to ensure said cooled heat-exchanging medium flowing to said catheter along said fourth path is at a stabilised cooled temperature.

3. The system of claim 1 wherein said cooling apparatus comprises a fan.

4. The system of claim 1 wherein said cooling apparatus comprises a peltier cooler.

5. The system of claim 1 wherein said pump comprises an electric pump connected to a power supply to circulate said heat-exchanging medium in said system.

6. The system of claim 1 wherein each of said first, second, third, fourth and fifth paths comprise thermo insulated flexible tubing.

7. The system of claim 1 wherein said heat-exchanging medium comprises water.

8. The system of claim 1 wherein said heat-exchanging medium comprises saline.

9. The system of claim 1 wherein said catheter is attached to a heat exchanging assembly with a body comprising:
a heat-exchanging element having a first end and a second end wherein said first end of said heat-exchanging element is an inlet for said heat-exchanging medium and said second end of said heat-exchanging element is an outlet for said heat exchanging medium;
a first temperature sensor to monitor the temperature of said heat-exchanging medium in said heat-exchanging element;
a second temperature sensor to monitor the temperature of a liquid flowing over said heat-exchanging element;
an inflation lumen having a first end and a second end wherein said first end is connected to an inflatable device located on a head-end of said catheter and said second end is connected to en inflation apparatus; and
a sensor lumen having a first end and a second end wherein said first end extends to a distal end of said catheter and said second end is connected to a sensing apparatus wherein said sensing apparatus is used to detect changes in the parameters at said distal end of said catheter.

10. The system of claim 9 wherein said first end of said sensor lumen extends to an opening in said distal end of said catheter wherein said opening allows bodily fluid to flow through said sensor lumen to said sensing apparatus.

11. A method for maintaining a heat-exchanging medium in a system at a constant temperature and at a constant flow rate comprising steps of:
pumping a heat-exchanging medium from a reservoir to a cooling apparatus;
cooling said heat-exchanging medium with said cooling apparatus;
changing a valve configuration between a first configuration and a second configuration wherein said valve is located between said cooling apparatus and said reservoir;
directing the flow of said cooled heat-exchanging medium from said cooling apparatus to said reservoir responsive to said valve being in said first configuration;
directing the flow of said tooled heat-exchanging medium from said cooling apparatus to a catheter responsive to said valve being in said second configuration; and
directing the flow of said cooled heat-exchanging medium from said catheter to said reservoir.

12. The method of claim 11 wherein said valve is located proximate said cooling apparatus to ensure said cooled heat-exchanging medium flowing to said catheter along said fourth path is at a stabilised cooled temperature.

13. The method of claim 11 wherein said heat-exchanging medium is selected from a group consisting of water or saline.

14. A heat exchanging assembly with a body adaptable to a catheter for measuring blood flow rate comprising:
a heat-exchanging element having a first end and a second end wherein said first end of said heat-exchanging element is an inlet for a heat-exchanging medium and said second end of said heat-exchanging element is an outlet for said heat exchanging medium;
a first temperature sensor to monitor the temperature of said heat-exchanging medium in said heat-exchanging element;
a second temperature sensor to monitor the temperature of a liquid flowing over said heat-exchanging element;
an inflation lumen having a first end and a second end wherein said first end is connected to an inflatable device located on a head-end of said catheter and said second end is connected to an inflation apparatus; and
a sensor lumen having a first end and a second end wherein said first end extends to a distal end of said catheter and said second end is connected to a sensing apparatus wherein said sensing apparatus is used to detect changes in the parameters at said distal end of said catheter.

15. The system of claim 14 wherein said first end of said sensor lumen extends to an opening in said distal end of said catheter wherein said opening allows bodily fluid to flow through said sensor lumen to said sensing apparatus.

16. The heat exchanging assembly of claim 14 wherein said sensing apparatus is used to determine the variation in blood pressure at said opening.

17. The heat exchanging assembly of claim 14 further comprising a system for providing said heat-exchanging medium to said first end of said heat-exchanging element at a constant temperature and constant flow rate.

18. The heat exchanging assembly of claim 14 wherein said heat-exchanging element is wrapped around said catheter in a helical configuration.

19. The heat exchanging assembly of claim 14 wherein said first temperature sensor is located proximate said first end of said heat-exchanging element.

20. The heat exchanging assembly of claim 14 further comprising a third temperature sensor to monitor the temperature of said heat exchanging medium wherein said third temperature sensor is located proximate said second end of said heat-exchanging element.

21. The heat exchanging assembly of claim 20 wherein one of said first, second and third temperature sensors are contact temperature sensors.

22. The heat exchanging assembly of claim 20 further comprising a temperature lumen wherein said temperature lumen is used to connect one of said first, second and third temperature sensors to a temperature monitoring apparatus.

23. The heat exchanging assembly of claim 22 wherein one of said heat-exchanging element, said pressure lumen, said sensor lumen and said temperature lumen are made of bio-compatible materials.

24. The heat exchanging assembly of claim 22 wherein one of said heat-exchanging element, said pressure lumen, said sensor lumen and said temperature lumen are made of thermo conductive materials.

25. The heat exchanging assembly of claim 14 wherein said heat exchanging medium is a liquid.

26. The heat exchanging assembly of claim 14 wherein said inflatable device is made from bio-compatible material.
